SEQUENCE LISTING (20 sequences)

SEQ ID NO: 1 - protein, 431 aa, synthetic construct
SFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGGGGSGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHD

SEQ ID NO: 2 - DNA, 1293 bp, synthetic construct
  source 1..1293: mol_type = other DNA
tccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggcggaggcggaagcggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgat

SEQ ID NO: 3 - DNA, 24 bp, synthetic construct
  source 1..24: mol_type = other DNA
cttggatcctccttcactgtagaa

SEQ ID NO: 4 - DNA, 27 bp, synthetic construct
  source 1..27: mol_type = other DNA
gcttccgcctccgccacaaacagttgc

SEQ ID NO: 5 - DNA, 27 bp, synthetic construct
  source 1..27: mol_type = other DNA
ggcggaggcggaagcggtacaatcact

SEQ ID NO: 6 - DNA, 43 bp, synthetic construct
  source 1..43: mol_type = other DNA
gtctcaagcttatggtgatggtgatgatgatcatgacaaatgg

SEQ ID NO: 7 - DNA, 663 bp, synthetic construct
  source 1..663: mol_type = other DNA
tccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgt

SEQ ID NO: 8 - DNA, 615 bp, synthetic construct
  source 1..615: mol_type = other DNA
ggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgat

SEQ ID NO: 9 - protein, 292 aa, synthetic construct
CVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSF

SEQ ID NO: 10 - protein, 221 aa, synthetic construct
SFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVC

SEQ ID NO: 11 - protein, 171 aa, synthetic construct
APATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQ

SEQ ID NO: 12 - protein, 199 aa, synthetic construct
ARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTI

SEQ ID NO: 13 - protein, 205 aa, synthetic construct
GTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHD

SEQ ID NO: 14 - protein, 172 aa, synthetic construct
TYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 15 - protein, 278 aa, synthetic construct
GSDLDRCTTFDDVQAPNYTQHTSSMRGVYYPDEIFRSDTLYLTQDLFLPFYSNVTGFHTINHTFDNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDTWGTSAAAYFVGYLKPTTFMLKYDENGTITDAVDCSQNPLAELKCSV

SEQ ID NO: 16 - protein, 196 aa, synthetic construct
VVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPA

SEQ ID NO: 17 - protein, 161 aa, synthetic construct
NAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSL

SEQ ID NO: 18 - protein, 200 aa, synthetic construct
LRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTAT

SEQ ID NO: 19 - protein, 205 aa, synthetic construct
GTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHE

SEQ ID NO: 20 - protein, 253 aa, synthetic construct
RLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLC

(19) United States
(12) Patent Application Publication
(10) Pub. No.: US 2025/0144062 A1
(43) Pub. Date: May 8, 2025

(54) BETA-CORONAVIRUS FUSION RECOMBINANT PROTEIN, AND PREPARATION METHOD AND APPLICATION THEREOF

(71) Applicant: BIOPHARMAGEN CORP., FANGZHOU SUZHOU, Suzhou (CN)

(72) Inventors: Yongping JIANG, Suzhou (CN); Hanlu WANG, Suzhou (CN); Wenhong JIANG, Suzhou (CN)

(73) Assignee: BIOPHARMAGEN CORP., FANGZHOU SUZHOU, Suzhou (CN)

(21) Appl. No.: 18/835,420

(22) PCT Filed: Jan. 10, 2023
(86) PCT No.: PCT/CN2023/071585

§ 371 Date: Aug. 2, 2024

(30) Foreign Application Priority Data
Feb. 8, 2022 (CN) 202210117774.7

(51) Int. Cl. A61K 31/215; C07K 1/36; C07K 14/005; C07K 16/10; C12N 15/62; C12N 15/70

(52) U.S. Cl. CPC A61K 31/215; C07K 1/36; C07K 14/005; C07K 16/1003; C12N 15/62; C12N 15/70; C07K 2319/00; C12N 2770/20022; C12N 2770/20034; C12N 2770/20051

(57) ABSTRACT

Disclosed is a betacoronavirus fusion recombinant protein, comprising an RBD region and a COVID19-SF5 fragment of a spike protein of SARS-COV-2 (COVID-19), and an amino acid sequence of the COVID19-SF5 fragment is an 880th amino acid to a 1084th amino acid of the S protein of the novel coronavirus COVID-19. According to the invention, a constant conserved fragment (COVID19-SF5) and a receptor binding domain (RBD) fragment are fused and expressed to provide a more-effective constant universal vaccine candidate recombinant fusion protein for such type of coronavirus, thus providing broader and better protection measures from two standpoints of inhibiting receptor recognition and providing universal protection.

### TECHNICAL FIELD

The present invention belongs to the field of biology, and more particularly, relates to a betacoronavirus fusion recombinant protein, and a preparation method and application thereof.

### BACKGROUND

Since the terminal of 2019, novel coronavirus (SARS-COV-2) infectious pneumonia (COVID-19) has been gradually prevalent from all over China and all over the world, and quickly spread to whole China and the whole world. With the exception of China, there have been serious epidemics all over the world, resulting in serious health problems around the world. Up to now, the epidemic situation is still very severe. Novel coronavirus and atypical pneumonia virus (SARS-COV) and Middle East Respiratory Syndrome Virus (MERS-COV) all belong to betacoronavirus, and can cause extremely serious respiratory syndrome. Although many vaccines have been marketed at present, due to a high mutation rate of the novel coronavirus, effects of different vaccines in preventing infection have all been reduced to varying degrees. Particularly, a recently emerged Omicron strain has a strong immune escape ability, which can not only escape from most therapeutic monoclonal antibodies, but also avoid the antibody immunity generated by the marketed vaccines to a great extent. The latest research reports also show that the Omicron strain has a new way to invade cells. In addition, there is still a risk of outbreak of other novel coronaviruses in the future, so that it is urgent to develop a universal preventive and efficient vaccine against such type of coronaviruses (comprising the prevalent novel coronavirus, Delta strain and Omicron strain, and possible coronaviruses in the future).

The novel coronavirus vaccines currently under research mainly comprise an inactivated vaccine, an adenovirus vector vaccine, a nucleic acid vaccine (mRNA vaccine), an attenuated live vaccine, and the like, and these vaccines generally have the shortcomings of insufficient specific immunogenicity, great difference in protection effect among people, enhancement of antibody-dependent infection and to-be-considered safety. In addition, in the face of the rapid variation of the novel coronavirus, such as the Delta and Omicron variants widely appearing at present, specific action time and effects of the vaccines are greatly limited. At present, a number of confirmed cases suffering from the novel coronavirus has exceeded 300 million, and in view of the high infectivity and mutation of the novel coronavirus, it is urgent to find a universal vaccine prevention and treatment drug against various coronaviruses and their variants.

S (Spike) protein plays an important role in the combination and invasion of coronavirus. The S protein is located on a surface of the coronavirus, and constitutes a unique spike structure on the surface of the virus, and the S protein consists of two subunits S1 and S2, wherein the S1 forms a spherical head of the spike protein, comprises large receptor binding domains (an N-terminal structural domain NTD and a receptor binding domain RBD) of the S protein, and is responsible for recognizing a host cell receptor, while the S2 forms a stem of the spike protein and participates in a membrane fusion process. The S2 subunit contains three functional domains, comprising a fusion peptide (FP) and peptide repetitive sequences (HR1 and HR2), and after the RBD at the tip of the S1 binds to the receptor, the FP in the S2 is inserted into a host cell membrane to change the conformation, which stimulates the HR1 and the HR2 to form a six-helix bundle (6HB), resulting in the fusion of virus membrane and cell membrane.

The S protein has the activity of receptor binding and membrane fusion to human upper respiratory tract cells, and is a key protein for mediating such type of virus to recognize and infect human cells. CN113943375A discloses a recombinant fusion protein from HR region of S2 protein of novel coronavirus and an application thereof. Such type of novel coronavirus recombinant fusion protein is a recombinant fusion protein obtained by ligating two conserved amino acid sequences HR1 and HR2 related to membrane fusion of the membrane protein S2 protein of the novel coronavirus through a linker peptide. The recombinant fusion protein may be induced to be expressed in Escherichia coli, with a high expression level, and is easy to be purified. The novel coronavirus recombinant fusion protein provided by the invention may form and maintain a stable trimer structure, simulate the conformation of an intermediate state of the membrane fusion of the novel coronavirus, and serve as a detection raw material for detecting a membrane fusion process of the novel coronavirus; and has good anti-novel coronavirus activity and good immunogenicity, and broad application prospects in the development of a protein drug for preventing or treating the novel coronavirus and the development of a novel coronavirus vaccine and an anti-novel coronavirus antibody.

CN112409469B discloses a fusion protein for transmembrane expression of a novel coronavirus antigen S2, a recombinant vector, a recombinant dendritic cell and applications thereof, and belongs to the technical field of whole-cell vaccines, wherein the fusion protein comprises a CD4 signal peptide, a protein of the novel coronavirus antigen S2, a Flag tag sequence and a CD4 transmembrane domain which are sequentially ligated; and in the invention, the S2 is subjected to transmembrane cell expression separately, which avoid a possible ADE risk caused by other S protein epitopes, and a cell vaccine constructed by the fusion protein provided by the invention may induce a higher neutralizing antibody titer in mice.

However, a more effective universal preventive and efficient vaccine is still urgent.

### SUMMARY

An S protein is subjected to fragmented and recombinant expression by analyzing a homologous structure and a biological function of such type of S protein in an earlier stage to prepare a comprehensive serum IgG antibody library, and then an antibody capable of cross-reacting with S proteins of various coronaviruses and a corresponding constant conserved region protein fragment are screened out, wherein the fragment is COVID19-SF5, with a sequence of an 880th amino acid to a 1084th amino acid of the S protein of the novel coronavirus COVID-19, and specifically, the amino acid sequence of the fragment is (SEQ ID NO. SEQ ID NO. 13):

GTITSGWTFGAGAALQIPFAMQMAYRENGIGVTQNVLYENQKLIANQFNS
AIGKIQDSLSSTASALGKLQDVVNQNAQALNTLVKQLSSNFGAISSVLND
ILSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTAP
AICHD.

According to the present invention, a constant conserved fragment (COVID19-SF5) and a receptor binding domain (RBD) fragment are fused and expressed to obtain a betacoronavirus fusion recombinant protein, with an amino acid sequence shown in SEQ ID NO. 1, so as to provide a more-effective constant universal vaccine candidate recombinant fusion protein for such type of coronavirus, thus providing broader and better protection measures from two standpoints of inhibiting receptor recognition and providing universal protection.

An RBD region of the S protein of the novel coronavirus COVID-19 is a COVID19-SF2 fragment (SEQ ID NO. 10), with an amino acid sequence of an 305^{th} amino acid to a 525^{th} amino acid of the S protein of the novel coronavirus COVID-19. The RBD region of the present invention is mainly an RBD of a 335^{th} site to a 522^{nd} site disclosed in a reference (Wrapp D, Wang N, Corbett K S, Goldsmith J A, Hsieh C-L, Abiona O, et al. Cryo-EM structure of the 2019-nCOV spike in the prefusion conformation. Science. 2020; 367 (6483): 1260-3), and the COVID19-SF2 of the present application contains the RBD region, and has certain overlapped parts with front and back regions at the same time.

The amino acid sequence of the fusion recombinant protein is shown in SEQ ID NO. 1. Specifically, an amino acid sequence of COVID19-SF2+5 is as follows:

SFTVEKGIYQTSNFRVQPTESIVRFPNITNLCPFGEVFNATRFASVYAWN
RKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYR
LFRKSNLKPFERDISTEIYQAGSTPCNGVEGENCYFPLQSYGFQPTNGVG
YQPYRVVVLSFELLHAPATVCGGGGSGTITSGWTFGAGAALQIPFAMQMA
YRENGIGVTQNVLYENQKLIANQFNSAIGKIQDSLSSTASALGKLQDVVN
QNAQALNTLVKQLSSNFGAISSVLNDILSRLDKVEAEVQIDRLITGRLQS
LQTYVTQQLIRAAEIRASANLAATKMSECVLGQSKRVDFCGKGYHLMSFP
QSAPHGVVFLHVTYVPAQEKNFTTAPAICHD.

The present invention further provides a gene encoding the fusion recombinant protein above. Preferably, a nucleotide sequence of the gene is SEQ ID NO. 2.

The present invention further provides a recombinant vector, which comprises the gene encoding the fusion recombinant protein above and a vector. The vector may be a pET series vector, a mammalian expression vector pcDNA3 series, and the like. In a specific embodiment, the present application adopts an expression vector pQE-3.

Further, the present invention further provides a recombinant bacterium, which comprises the recombinant vector above. A host bacterium may be Escherichia coli BL21 and M15, an insect cell sf9, mammalian cells CHO and 293, and the like.

The present invention further provides applications of the fusion recombinant protein above, the gene encoding the fusion recombinant protein, the recombinant vector and the recombinant bacterium in preparing a universal vaccine and a universal antibody of betacoronavirus.

In a specific embodiment, the expression strain construction and protein expression purification of the fusion protein of the COVID19-SF2 protein fragment and the COVID19-SF5 protein fragment of the SARS-COV-2 are realized by the following method.
1) A SARS-COV-2 full-length DNA is used as a template, different PCR primers are designed for a COVID19-SF2 protein fragment and a COVID19-SF5 protein fragment, a BamH I enzyme cutting site is introduced at a 5′ terminal of the COVID19-SF2 protein fragment and a reverse complementary sequence of a flexible linker peptide is introduced at a 3′ terminal of the COVID19-SF2 protein fragment, and a sequence of the flexible liner peptide is introduced at a 5′ terminal of the COVID19-SF5 protein fragment, a Hind III enzyme cutting site is introduced at a 3′ terminal of the COVID19-SF5 protein fragment and a 6×His encoding gene is introduced at a C terminal of the COVID19-SF5 protein. PCR is carried out on the COVID19-SF2 protein fragment and the COVID19-SF5 protein fragment respectively first, and after amplification, PCR products are verified by 2% agarose gel electrophoresis. The PCR products are purified with a PCR product purification kit.
2) Overlap extension PCR is carried out on the PCR products of the COVID19-SF2 protein fragment and the COVID19-SF5 protein fragment respectively amplified in the first step, a fusion protein expression gene is obtained by ligation, and after amplification, PCR products are verified by 1% agarose gel electrophoresis. The PCR products are purified with a PCR product purification kit.
3) The target gene is ligated with the expression vector pQE-3 through the BamH I and Hind III enzyme cutting sites at the 5′ terminal and the 3′ terminal of the sequences. An enzyme cutting product is verified by 1% agarose gel electrophoresis. The vector and the target gene respectively recover and purify the enzyme cutting product with a gel recovery and purification kit. After purification, a nucleic acid concentration is detected with a One drop spectrophotometer.
4) Numbers of target gene fragments and plasmids in an enzyme-linked system are calculated according to a molar ratio of a target gene fragment to a pQE-3 plasmid vector of 4:1. The enzyme ligation is carried out at 4° C. overnight. An obtained enzyme-linked product is an expression vector containing the fusion protein gene.
5) Transformation: the expression vector containing the fusion protein gene is transformed into an Escherichia coli M15 strain by a competent cell preparetion method.
6) Selection of positive clones: strains growing on a selective plate are selected for colony PCR, and protein expression of strains positive for PCR is induced.
7) Expression induction: the clones with positive colony PCR are taken for expanded culture by a specific method that: the positive clones on the plate are selected for overnight culture, and overnight bacteria are added with fresh culture medium for expanded culture for about 4 hours, and then added with IPTG with a final concentration of 100 mM to induce expression for 4 hours. Bacterial precipitates are acquired by centrifugation, and protein expression is verified by SDS-PAGE.
8) Acquisition of inclusion bodies for purification and folding: expression bacteria are acquired and lysed to acquire the recombinant fusion protein inclusion bodies, which are dissolved in 6 M guanidine hydrochloride solution (0.05 mol/L tris, 5 mmol/L EDTA, 6 mol/L guanidine hydrochloride, 1% β-mercaptoethanol, pH 8.0), wherein 1 g of inclusion bodies are dissolved in 100 mL of 6 M guanidine hydrochloride.
Purification with a Ni-NTA affinity column: the column is loaded according to steps suggested by a manufacturer of the Ni-NTA affinity column, then the affinity column is balanced with 8 M urea (5 column volumes, dissolved in a phosphate buffer, pH 8.0), a solution of the inclusion bodies dissolved in guanidine hydrochloride is loaded at a speed of 5 mL/min, a impurity protein is eluted with sodium phosphate (5 column volumes) at pH 6.0 after loading, and then a target protein is collected with an acetic acid at pH 4.5.
Folding steps of dialysis with urea gradient solution: the purified protein solution above is diluted to 0.3 mg/mL with 3 M urea (contained in a sodium acetate buffer, PH 4.5), and dialyzed with urea dialysates with different concentrations once sequentially at 4° C. for 24 hours each time, wherein a ratio of an internal liquid to an external liquid of a dialysis bag is 1:5, the internal liquid is 3.5 M urea-sodium acetate buffer, and the external liquids are dialysis buffers of 3 M, 2.5 M, 1.5 M, 1 M, 0.5 M, 0 M and 0 M urea sequentially.
9) Acquisition of recombinant fusion protein: a target fusion protein solution is centrifuged at 15000 rpm for 20 minutes after dialysis, a protein concentration is determined by the Braford method, and the solution is subjected to filter sterilization with a 0.22 μm filter membrane, added with mannitol, and then stored in a refrigerator at −80° C.

By the above method, an efficient and universal coronavirus fusion protein named “COVID19-SF2+5” is obtained, and an antibody of the fusion protein has certain cross-reactivity with each S protein fragment, and particularly has a strong binding ability to the COVID19-SF2 and the COVID19-SF5. It is suggested that the fusion protein not only retains the RBD region, but also can induce the generation of an IgG antibody specifically blocking the binding between the virus and the receptor, and the fusion protein comprises the constant conserved fragment COVID19-SF5 at the same time, which can induce the generation of more extensive broad-spectrum IgG antibodies cross-reacting with multiple S protein fragments.

Further, an antiserum of the “COVID19-SF2+5” fusion protein is obtained by mouse immunization, and a serum comprehensive antibody IgG is obtained by purification, so that the serum comprehensive antibody IgG of the COVID19-SF2+COVID19-SF5 fusion protein of the S protein of the novel coronavirus SARS-COV-2 is prepared.

The present invention further provides a preparation method of the recombinant fusion protein by industrial fermentation, which comprises the following steps of:
(1) using the recombinant bacterium as a seed strain, and amplifying the seed strain by overnight shaking as a seed solution;
(2) subjecting the seed solution to fermentation culture in a 2×YT culture medium, collecting fermented bacteria by an industrial automatic continuous centrifuge, preparing the collected bacteria into a suspension with an extracting solution A, lysing with a hydrolyase, then processing with an extracting solution B, collecting precipitated inclusion bodies after centrifugation, diluting with a buffer, and then centrifuging, wherein precipitates are insoluble inclusion bodies;
(3) further dissolving the inclusion bodies in a buffer, centrifuging and collecting supernatant for ultrafiltration and concentration to obtain a concentrated sample, then allowing the concentrated sample to pass through a Ni-NTA affinity column, purifying on an AKTA protein purification system, and collecting the sample; and
(4) dialyzing the collected sample in a chromatographic cabinet at 4° C., centrifuging the dialyzed sample to take a supernatant, concentrating the supernatant by an ultrafiltration concentrator, and then purifying by Sephadex G-75 chromatography on the AKTA protein purification system; and collecting a sample according to a protein peak of the AKTA protein purification system to obtain a purified fusion protein.

In a specific embodiment, the preparation method comprises the following steps:
1) Cleaning is carried out before fermentation and inoculation, sterile operation is concerned, and other strains should not be fermented in the same fermentation time.
2) A fermentor and a pipeline are sterilized, before each fermentation, an empty fermentor is sterilized at 121° C. for 30 minutes, the fermentor is sterilized again at 121° C. for 30 minutes after a prepared culture medium is put into the fermentor, and a seed solution (previously frozen dominant expression strains, wherein the seed strain are amplified by overnight shaking) is inoculated when the fermentor is cooled to a required temperature of 37° C.; and 500 mL of seed solution and 35000 mL of culture medium are added to the 40 L fermentor (with overnight bacteria as the seed solution and a 2×YT culture medium as the culture medium), wherein the 2×YT culture medium is prepared by: adding 16 g of tryptone, 10 g of yeast extract and 5 g of sodium chloride into 1 L of culture medium, evenly stirring the mixture, and then sterilizing the mixture at a high temperature and a high pressure.
3) Fermentation conditions, such as a temperature, a pH value, an oxygen flow and fermentation time, are controlled by a computer operating system of the fermentor. The temperature is set to be 37° C., the pH value is set to be 7.0, and the fermentation time is set to be about 7 hours. (A dissolved oxygen value or dissolved oxygen concentration (DO value) of 60%, a temperature of 37° C., a pH value of 7.0, the addition of an inducer IPTG when a bacterial concentration reaches a peak value, and total culture time of 7 hours.)
4) The fermented bacteria are collected by an industrial automatic continuous centrifuge at a centrifugal speed of 10000 g and a temperature of 4° C. for 1 hour.
5) Every 40 g of the acquired bacteria are added with 1000 mL of extracting solution A (50 mM Tris, pH 8.0, containing 1.5 mm EDTA) to prepare a suspension, and then lysed with 250 mg of lysozyme.
6) A lysate is processed with an extracting solution B (1.5 M NaCl, 100 mM CaCl₂), 100 mM MgCl₂, 0.002% DNase I). Every 1000 mL of the above lysate is added with 100 mL of extracting solution B.
7) After the mixture is centrifuged at 10000 g and 4° C. for 10 minutes, precipitated inclusion bodies are collected and evenly suspended in 50 mM phosphate buffer (containing 0.15 M sodium chloride and 4 M urea, pH 7.0). 1 g of precipitates are added with 10 mL of buffer and centrifuged at 10000 g and 4° C. for 10 minutes, and precipitates are insoluble inclusion bodies, which may be collected and stored at −80° C. for half a year.

Folding and purification of protein:
1) Every 10 g of crude purified inclusion bodies are dissolved in 2000 mL of buffer (0.1 M Tris-HCl, pH 7.5 containing 6 M guanidine hydrochloride, 20 mM DTT, and 20 mM EDTA), and stirred at 20° C. for 1 hour.
2) The mixture is centrifuged at 10000 g and 4° C. for 30 minutes, and then a supernatant is taken for ultrafiltration and concentration. Every 2000 mL of the supernatant is concentrated into about 200 mL.
3) A concentrated sample passes through a Ni-NTA affinity column, and is purified on an AKTA protein purification system. Specific operation steps are the same as those in the above description.
4) The collected sample is dialyzed in a chromatographic cabinet at 4° C. according to the above method.
5) The dialyzed sample is centrifuged at 10000 g and 4° C. for 30 minutes, and then a supernatant is taken.
6) The supernatant is concentrated by an ultrafiltration concentrator, and every 2000 mL of the supernatant is concentrated into 300 mL.
7) The sample is purified by Sephadex G-75 chromatography on the AKTA protein purification system, with a column length of 1.2 m, a diameter of 4 cm, and a flow rate of 1 mL/min.
8) A sample is collected according to a protein peak of the AKTA protein purification system to obtain a purified fusion protein.

Sterilization and endotoxin removal of protein:
1) An endotoxin of the purified fusion protein is removed by a Polymyxin (Bio-rad) chromatographic column.
2) An endotoxin-removed sample is subjected to filtration sterilization by a 0.22 μm sterile filter, and then subpackaged and stored at 4° C.

Beneficial effects: different from the previous research methods, the present invention combines the previous researches in the laboratory, takes the structural and functional analysis of the S protein of the betacoronavirus as a breakthrough point, carries out amino acid sequence regionalization and linear homology matching analysis of various coronavirus proteins, carries out fragmented expression through a homology structure of the S protein to establish a recombinant protein fragment library covering the whole region of the S protein, and finds the COVID19-SF5 fragment of the S protein of the SARS-COV-2, which has universal cross-reaction with various fragments of the S protein of the SARS-COV and various fragments of the S protein of the SARS-COV-2 through cross-reaction between a serum antibody library obtained from immunized mice and various S protein fragments, and other researches. According to the present invention, the COVID19-SF5 protein fragment with universal cross-reactivity and the COVID19-SF2 protein fragment containing the virus receptor binding domain (RBD) are ligated through the flexible linker peptide Gly4Ser to form the fusion protein with a multifunctional effect, and the serum IgG antibodies are obtained by immunizing the mouse with the fusion protein. Tests show that a binding ability of the fusion protein to cells is significantly higher than that of the COVID19-SF2 or COVID19-SF5 protein fragment alone, and the serum IgG antibody can cross-react with various S protein fragments, and can significantly inhibit pseudovirus from infecting cells. To sum up, a universal fusion protein vaccine has obvious advantages:
(1) by a gene recombination technology, the product of the present invention is the fusion protein obtained by ligating two targeted specific fragments of the S protein of the virus, without involving genes, other virus vectors and inactivated viruses in entering the human body, and the product has a clear quality control and quality assurance system, thus ensuring the safety of the product;
(2) by studying the structure and function of the S protein, the constant conserved universal protein domain and the receptor binding domain of the betacoronavirus are ligated, which can strongly stimulate the response of a human immune system, thus producing an IgG neutralizing antibody capable of effectively preventing virus infection and a specific antibody capable of recognizing universal epitopes of the betacoronavirus; and
(3) the natural evolution and variation of any virus will help the virus to infect a host more easily and coexist with the host for a long time, various functions of the virus are manifested in a protein of the virus or protein regulation of the host, many aspects of functions of the fusion protein of the S protein of the novel coronavirus still need to be studied and clarified, and the effective antibody generated in the mouse by the fusion protein fragment in this project has functional specificity.

### BRIEF DESCRIPTION OF THE DRAWINGS

FIG. 1 is a schematic diagram of plasmid construction verified by double-enzyme cutting;

FIG. 2 shows expression of a recombinant fusion protein identified by SDS-PAGE; and

FIG. 3 shows a binding ability of three protein fragments to Vero-E6 cells.

### DETAILED DESCRIPTION

The present invention is further described in detail hereinafter with reference to specific embodiments, and the embodiments will help to understand the present invention, but the scope of protection of the present invention is not limited to the following embodiments.

Embodiment 1: Expression strain construction and protein expression purification of fusion protein “COVID19-SF-2+5” of COVID19-SF2 protein fragment and COVID19-SF5 protein fragment of SARS-COV-2
1) A full-length DNA of SARS-COV-2 was used as a template, different PCR primers were designed for the COVID19-SF2 protein fragment and the COVID19-SF5 protein fragment, a BamH I enzyme cutting site was introduced at a 5′ terminal of the COVID19-SF2 protein fragment and a reverse complementary sequence of a flexible linker peptide was introduced at a 3′ terminal of the COVID19-SF2 protein fragment, and a sequence of the flexible liner peptide was introduced at a 5′ terminal of the COVID19-SF5 protein fragment, wherein the flexible liner peptide was Gly4Ser, a Hind III enzyme cutting site was introduced at a 3′ terminal of the COVID19-SF5 protein fragment and a 6×His encoding gene was introduced at a C terminal of the COVID19-SF5 protein. PCR was carried out on the COVID19-SF2 protein fragment and the COVID19-SF5 protein fragment respectively first, wherein a PCR system and amplification conditions were as follows:

TABLE 1-1
Sequences of PCR primers

| | | |
|---|---|---|
| COVID19-SF2 | 5′-CTTGGATCCTCCTTCA | SEQ ID |
| forward | CTGTAGAAA-3′ | NO.3 |
| primer | | |
| COVID19-SF2 | 5′-GCTTCCGCCTCCGCCA | SEQ ID |
| reverse | CAAACAGTTGC-3′ | NO.4 |
| primer | | |
| COVID19-SF5 | 5′-GGCGGAGGCGGAAGCG | SEQ ID |
| forward | GTACAATCACT-3′ | NO.5 |
| primer | | |
| COVID19-SF5 | 5′-GTCTCAAGCTTATGGT | SEQ ID |
| reverse | GATGGTGATGATGATCATG | NO.6 |
| primer | ACAAATGG-3′ | |

TABLE 1-2
PCR system

| | |
|---|---|
| Template | 1 μL (10-100 ng) |
| Forward primer | 2 μL |
| Reverse primer | 2 μL |
| 10 × pFu buffer | 5 μL |
| dNTP (10 mM) | 1 μL |
| pFu enzyme | 1 μL |
| ddH2O | 38 μL |
| Total volume | 50 μL |

The amplification conditions comprised: 94° C., 30 seconds; 56° C., 1 minute; 72° C., 1 minute and 30 seconds; and 35 cycles.

After amplification, PCR products were verified by 2% agarose gel electrophoresis. The PCR products were purified with a PCR product purification kit. A gene sequence of the COVID19-SF2 was shown in SEQ ID NO. 7, and a gene sequence of the COVID19-SF5 was shown in SEQ ID No. 8.
2) Overlap extension PCR was carried out on the PCR products of the COVID19-SF2 protein fragment and the COVID19-SF5 protein fragment respectively amplified in the first step, and a fusion protein expression gene was obtained by ligation, wherein a PCR system and amplification conditions were as follows:

TABLE 2
PCR system

| | |
|---|---|
| Template | 1 μL (1-100 ng) + |
| | 1 μL (1-100 ng) |
| COVID19-SF2 forward primer | 2 μL |
| COVID19-SF5 reverse primer | 2 μL |
| 10 × pFu buffer | 5 μL |
| dNTP (10 mM) | 1 μL |
| pFu enzyme | 1 μL |
| ddH2O | 37 μL |
| Total volume | 50 μL |

The amplification conditions comprised: 94° C., 30 seconds; 56° C., 1 minute; 72° C., 2 minutes and 40 seconds; and 30 cycles.

After amplification, PCR products were verified by 1% agarose gel electrophoresis. The PCR products were purified with a PCR product purification kit.
3) Enzyme cutting: the target gene (SEQ ID NO. 2) was ligated with the expression vector pQE-3 through the BamH I and Hind III enzyme cutting sites at the 5′ terminal and the 3′ terminal of the sequences. An enzyme cutting system was as follows:

TABLE 3
Vectors

| | |
|---|---|
| Hind III | 2.5 μL |
| BamH I | 2.5 μL |
| 10 × K buffer | 5 μL |
| Vector | 2.5 μg |
| [symbol] DW | ~ |
| Total volume | 50 μL |

TABLE 4
Target genes

| | |
|---|---|
| Hind III | 2.5 μL |
| BamH I | 2.5 μL |
| 10 × K buffer | 5 μL |
| Target DNA | 42 μL (2.5 μg) |
| Total volume | 50 μL |

The enzyme cutting was carried out in a water bath at 37° C. for 0.5 hour. An enzyme cutting product was verified with 1% agarose gel. The vector and the target gene respectively recovered and purified the enzyme cutting product with a gel recovery and purification kit. After purification, a nucleic acid concentration was detected with One drop.
4) Enzyme ligation

Numbers of target gene fragments and plasmids in an enzyme-ligated system were calculated according to a molar ratio of a target gene fragment to a pQE-3 plasmid vector of 4:1. The enzyme-ligated system was as follows:

TABLE 5

| | |
|---|---|
| PQE-3 plasmid | 1 μL |
| T4 buffer | 2 μL |
| Target DNA | 1 μL |
| T4 ligase | 1 μL |
| ddH2O | 16 μL |
| Total volume | 20 μL |

The enzyme ligation was carried out at 4° C. overnight.
An obtained enzyme-ligated product was an expression vector containing the fusion protein gene.

5) Transformation

The expression vector containing the fusion protein gene was transformed into an Escherichia coli M15 strain by a competent method.
6) Selection of positive clones

Strains growing on a selective plate were selected for colony PCR, and protein expression of strains positive for PCR was induced.
7) Expression induction

The clones with positive colony PCR were taken for expanded culture by a specific method that: the positive clones on the plate were selected for overnight culture, and overnight bacteria were added with fresh culture medium for expanded culture for about 4 hours, and then added with IPTG with a final concentration of 100 mM to induce expression for 4 hours. Bacterial precipitates were acquired by centrifugation, and protein expression was verified by SDS-PAGE.
8) Acquisition of inclusion bodies for purification and folding

Expression bacteria were acquired and lysed to acquire the recombinant fusion protein inclusion bodies, which were dissolved in 6 M guanidine hydrochloride solution (0.05 mol/L tris, 5 mmol/L EDTA, 6 mol/L guanidine hydrochloride, 1% β-mercaptoethanol, pH 8.0), wherein 1 g of inclusion bodies were dissolved in 100 mL of 6 M guanidine hydrochloride.

Purification with a Ni-NTA affinity column: the column was loaded according to steps suggested by a manufacturer of the Ni-NTA affinity column, then the affinity column was balanced with 8 M urea (5 column volumes, dissolved in a phosphate buffer, pH 8.0), a solution of the inclusion bodies dissolved in guanidine hydrochloride was loaded at a speed of 5 mL/min, a impurity protein was eluted with sodium phosphate (5 column volumes) at pH 6.0 after loading, and then a target protein was collected with an acetic acid at pH 4.5.

Folding steps of dialysis with urea gradient solution: the purified protein solution above was diluted to 0.3 mg/mL with 3 M urea (contained in a sodium acetate buffer, PH 4.5), and dialyzed with urea dialysates with different concentrations once sequentially at 4° C. for 24 hours each time, wherein a ratio of an internal liquid to an external liquid of a dialysis bag was 1:5, the internal liquid was 3.5 M urea-sodium acetate buffer, and the external liquids ware dialysis buffers of 3 M, 2.5 M, 1.5 M, 1 M, 0.5 M, 0 M and 0 M urea sequentially.
9) Acquisition of fusion protein

A target protein solution was centrifuged at 15000 rpm for 20 minutes after dialysis, a protein concentration was determined by the Braford method, and the solution was subjected to filter sterilization with a 0.22 μm filter membrane, added with mannitol, and then stored in a refrigerator at −80° C.

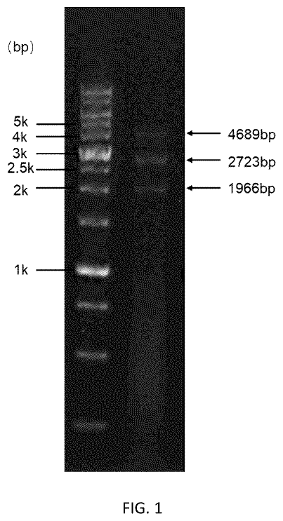

Plasmid construction verified by double-enzyme cutting referred to FIG. 1. Double enzyme cutting was carried out with a BamHI restriction endonuclease and a Ncol restriction endonuclease with a distance of 673 bp from a Hind III enzyme cutting site, and enzyme cutting results were shown in the figure below, wherein a full-length plasmid was 4689 bp, and two bands after double enzyme cutting were shown in agarose gel electrophoresis: the target gene plus 673 bp between Hind III and Ncol restriction endonucleases was 1966 bp, and the remaining vector was 2723 bp, which was consistent with the theory.

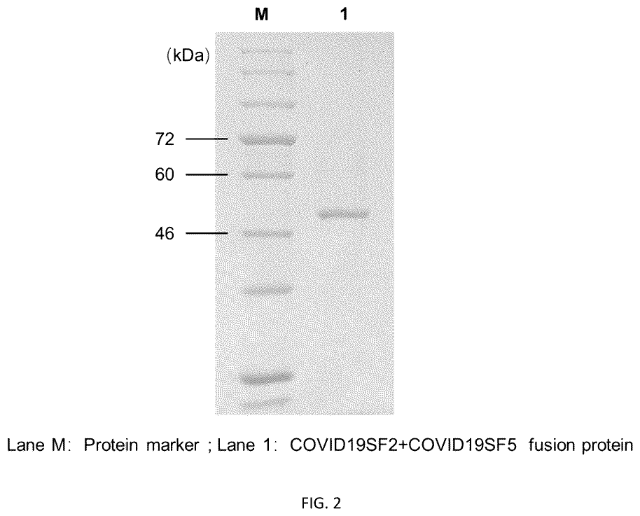

Protein expression was verified by SDS-PAGE, as shown in FIG. 2 below. It could be seen from the figure that a band position of the fusion protein was consistent with a theoretical band position of 48.2 kD, and a band of the protein purified by the Ni column was single, thus having good expression and folding effects.

### Embodiment 2: Identification of Universal Fusion Protein of Betacoronavirus

Specificity and universal cross-reactivity of a fusion protein antibody were detected by an ELISA method.
1) Determination of antibody titer: an immunized recombinant fusion protein was used as an antigen, a purified serum IgG antibody was diluted by multiple times and parallel wells were set up, then ELISA determination was carried out, an OD value was detected at 450 nm, and results were analyzed.

A titer of the comprehensive antibody IgG (50 μg/mL) was detected by ELISA six months after first immunization with the fusion protein (referring to Table 2), and mice showed a good immune effect on the fusion protein fragment, with an antibody titer still reaching 1:1600 six months after first immunization.

TABLE 6
Detection of titer of serum comprehensive antibody
IgG of fusion protein by ELISA

| Recombinant protein | Antibody titer |
|---|---|
| COVID-SF2 + 5 fusion protein | 1: 1600 |

2) Determination of universal cross-reactivity: 12 recombinant protein fragments were used as antigens, a fusion protein antibody reacted with the antigens, 3 parallel wells were set up for each group, ELISA determination was carried out, an OD value was detected at 450 nm, and results were analyzed. Starting-ending amino acid positions and corresponding amino acid sequences of the 12 recombinant protein fragments were shown in Table 7-1:

TABLE 7-1
12 S protein fragments containing overlapped domains

| | | Starting and | |
|---|---|---|---|
| | | ending amino | Amino acid |
| Protein fragment | | acid sites | sequence |
| | COVID19-SF1 | 15-306 | SEQ ID NO. 9 |
| SARS- | COVID19-SF2 | 305-525 | SEQ ID NO. 10 |
| CoV-2 | COVID19-SF3 | 520-690 | SEQ ID NO. 11 |
| | COVID19-SF4 | 684-882 | SEQ ID NO. 12 |
| | COVID19-SE5 | 880-1084 | SEQ ID NO. 13 |
| | COVID19-SF6 | 1066-1237 | SEQ ID NO. 14 |
| SARS- | SARS-SF1 | 15-292 | SEQ ID NO. 15 |
| CoV | SARS-SF2 | 315-510 | SEQ ID NO. 16 |
| | SARS-SF3 | 507-667 | SEQ ID NO. 17 |
| | SARS-SF4 | 668-867 | SEQ ID NO. 18 |
| | SARS-SF5 | 864-1068 | SEQ ID NO. 19 |
| | SARS-SF6 | 967-1219 | SEQ ID NO. 20 |

A binding ability of the comprehensive antibody IgG of the fusion protein to various fragments of the S protein was detected by ELISA, and it was found that the antibody had certain cross-reactivity with each fragment of the S protein, with a high binding ability. Although a reaction effectiveness was weakened six months after first immunization, the antibody still had obvious cross-reactivity with most protein fragments, which suggested that the fusion protein could not only generate a highly specific neutralizing antibody after immunizing mice, but also contain a variety of constant conserved specific protein fragments of the betacoronavirus. ELISA detection results referred to Table 7-2 and Table 7-3.

TABLE 7-2
Detection of cross-reaction between COVID19-SF2 + 5 fusion protein antibody and protein fragments

| (Cross- | | | | | | |
|---|---|---|---|---|---|---|
| reactivity) | Protein fragment library | | | | | |
| COVID19- | 2019-nCoV | | | | | |
| SF2 + 5 | COVID19-SF1 | COVID19-SF2 | COVID19-SF3 | COVID19-SF4 | COVID19-SF5 | COVID19-SF6 |
| Serum IgG | 1.55 | 0.99 | 0.45 | 0.16 | 0.6 | 0.25 |
| antibody | SARS | | | | | |
| (one month | SARS-SF1 | SARS-SF2 | SARS-SF3 | SARS-SF4 | SARS-SF5 | SARS-SF6 |
| after first | 0.82 | 0.36 | 0.45 | 0.25 | 0.54 | 0.51 |
| immunization) | | | | | | |

Test value = average value of (OD_{experimental group} − OD_{control group})

TABLE 7-3
Detection of cross-reaction between COVID19-SF2 + 5 fusion protein antibody and protein fragments

Cross-reaction
(cross-

| reactivity) | Protein fragment library | | | | | |
|---|---|---|---|---|---|---|
| COVID19- | 2019-nCoV | | | | | |
| SF2 + 5 | COVID-SF1 | COVID19-SF2 | COVID19-SF3 | COVID19-SF4 | COVID19-SF5 | COVID19-SF6 |
| Serum IgG | 0.23 | 0.17 | 0.14 | 0.16 | 0.06 | 0.15 |
| antibody | SARS | | | | | |
| (six months | SARS-SF1 | SARS-SF2 | SARS-SF3 | SARS-SF4 | SARS-SF5 | SARS-SF6 |
| after first | 0.19 | 0.15 | 0.09 | 0.15 | 0.23 | 0.14 |
| immunization) | | | | | | |

Test value = average value of (OD_{experimental group} − OD_{control group})

### Embodiment 3: Mouse Safety and Antibody Response Detections of Universol Specific Coronavirus Fusion Protein Vaccine

20 BALB/c mice were immunized with 0.20 mg/mL COVID19-SF2+5 fusion protein, the safety of the mice during injection was observed, and an IgG response level was detected on the 28^{th} and 45^{th} days.

The mouse safety and IgG response detections 45 days after injection of the COVID19-SF2+5 fusion protein were observed.

The 20 mice inoculated with the COVID19-SF2+5 fusion protein were in good health, and all of the mice could generate effective IgG antibodies. Results of the safety and IgG response detections were shown in Table 8 below:

TABLE 8
Safety and IgG response detections of COVID19-SF2 + 5 in mice

| | Injection time | | | | | |
|---|---|---|---|---|---|---|
| | Number of days | | | | | |
| Effect | 3 | 7 | 14 | 21 | 28 | 45 |
| Safety | All in | All in | All in | All in | All in | All in |
| | good | good | good | good | good | good |
| | health | health | health | health | health | health |
| IgG | | | | | ✓ | ✓ |
| response | | | | | | |

### Embodiment 4: Detection of Binding Ability of Universal Specific Coronavirus Fusion Protein COVID19-SF2+5 to Cells

African green monkey kidney cells (Vero-E6) were processed and counted, and then 1.5×10⁵ cells were resuspended with 100 μL of cell washing solution (PBS containing 1% BSA), and added with the universal specific coronavirus fusion protein with a final concentration of 2 μg/mL. Meanwhile, control tubes were added with the same molar weights of COVID19-SF2 and COVID19-SF5 for control study, fully mixed, and incubated at 37° C. for 1 hour. The reaction tubes were shaken every 10 minutes during incubation to make cells fully react with the proteins, then added with a proper amount of cell washing solution, centrifuged at 5000 rpm for 2 minutes, subjected to supernatant removal, washed twice, then added with a proper amount of fluorescent marked secondary antibody (anti-His Tag PE, Abcam, diluted by 1:50), fully mixed, and incubated at 4° C. for 1 hour in the dark. The reaction tubes were shaken every 10 minutes during incubation, added with a proper amount of cell washing solution, centrifuged at 5000 rpm for 2 minutes, subjected to supernatant removal, and washed twice. The cells were resuspended with 200 μL of cell washing solution, and a fluorescent signal on a cell surface was detected with a flow cytometer.

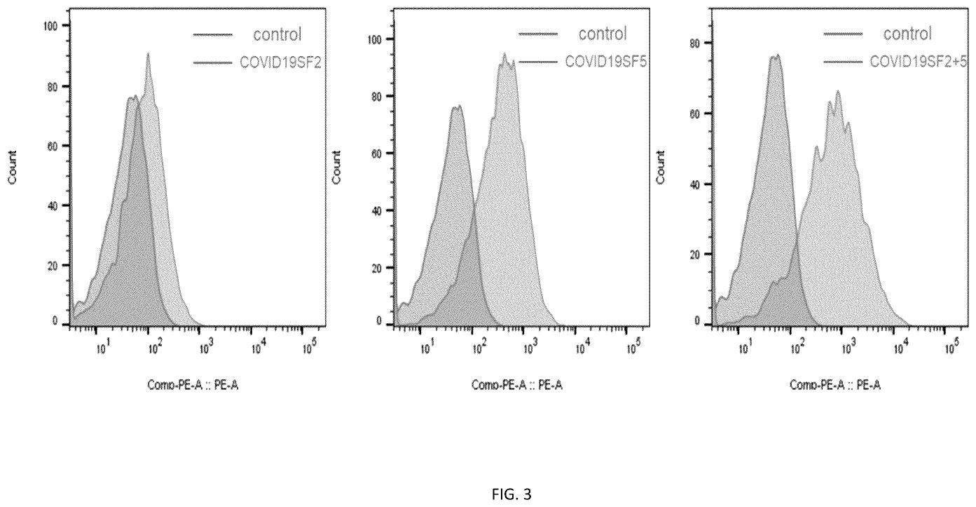

The universal specific coronavirus protein fusion protein COVID19-SF2+5 had a strong fluorescence shift after binding to Vero-E6 cells, as shown in FIG. 3. A binding ability of the fusion protein COVID19-SF2+5 to the Vero-E6 cells was higher than that of the simple COVID19-SF2 or COVID19-SF5 fragment. Specific cell and protein binding ratios referred to Table 9.

TABLE 9
Binding of Vero-E6 cells to COVID19-SF2 protein, COVID19-SF5
protein or COVID19-SF2 + COVID19-SF5 fusion protein

| | COVID19- | COVID19- | COVID19- |
|---|---|---|---|
| Protein fragment | SF2 | SF5 | SF2+5 |
| Binding ability (%) | 11.7 | 67.5 | 77.5 |

### Embodiment 5: Detection of Pseudovirus Inhibition Ability of Comprehensive Antibody IgG Corresponding to Universal Fusion Protein of Betacoronavirus

Expression of luciferase in cells infected by SARS-COV-2 pseudovirus was detected by a multifunctional microplate reader, so as to judge the pseudovirus inhibition ability of the comprehensive antibody corresponding to the universal fusion protein.

When hACE2-293T cells were taken as infected cells, the hACE2-293T cells were inoculated in a 96-well plate by 2×10⁴/well the night before. After 18 hours, 10 μg/mL antiserum IgG of the fusion protein was mixed with 650 TCID50/well pseudovirus, and then the mixture was added into the cells to be incubated for 48 hours. According to a scheme of a manufacturer, the expression of luciferase was measured by the multifunctional microplate reader with a luciferase detection kit to obtain an antiviral ability of the serum antibody. A cell control containing only cells and a virus control containing only viruses and cells were set in each plate. Three parallel experiments were set for each group. An inhibition rate of the serum antibody was calculated by taking an inhibition rate of the cell control containing only cells as 100% and taking an inhibition rate of the virus control containing viruses and cells as 0%.

Inhibition rates of the antiserum of the fusion protein on pseudovirus infection to cells were detected by pseudovirus neutralization experiments (referring to Table 10), which were results of three parallel experiments. It could be seen from the table that the serum IgG antibody generated by mice immunized with the fusion protein COVID19-SF2+5 could inhibit the pseudovirus infection to cells to some extent, with an inhibition rate of about 40%.

TABLE 10
Inhibiting effect of antibody of COVID19-SF2 + 5 fusion
protein on SARS-COV-2 pseudovirus

| Pseudovirus neutralization | | | |
|---|---|---|---|
| experiment | Inhibition rate (%) | | |
| Serum comprehensive | Numerical | Numerical | Numerical |
| antibody IgG | value 1 | value 2 | value 3 |
| of COVID19-SF2 + 5 | | | |
| fusion protein | 35.1 | 39.9 | 46.9 |

### Embodiment 6 Preparation of Recombinant Fusion Protein by Industrial Fermentation

1) Cleaning was carried out before fermentation and inoculation, sterile operation was concerned, and other strains should not be fermented in the same fermentation time.
2) A fermentor and a pipeline were sterilized, before each fermentation, an empty fermentor was sterilized at 121° C. for 30 minutes, the fermentor was sterilized again at 121° C. for 30 minutes after a prepared culture medium was put into the fermentor, and a seed solution (previously frozen dominant expression strains, wherein the seed strain were amplified by overnight shaking) was inoculated when the fermentor was cooled to a required temperature of 37° C.; and 500 mL of seed solution and 35000 mL of culture medium were added to the 40 L fermentor (with overnight bacteria as the seed solution and a 2×YT culture medium as the culture medium), wherein the 2×YT culture medium was prepared by: adding 16 g of tryptone, 10 g of yeast extract and 5 g of sodium chloride into 1 L of culture medium, evenly stirring the mixture, and then sterilizing the mixture at a high temperature and a high pressure.
3) Fermentation conditions, such as a temperature, a pH value, an oxygen flow and fermentation time, were controlled by a computer operating system of the fermentor. The temperature was set to be 37° C., the pH value was set to be 7.0, and the fermentation time was set to be about 7 hours. (A dissolved oxygen value or dissolved oxygen concentration (DO value) of 60%, a temperature of 37° C., a pH value of 7.0, the addition of an inducer IPTG when a bacterial concentration reaches a peak value, and total culture time of 7 hours.)
4) The fermented bacteria were collected by an industrial automatic continuous centrifuge at a centrifugal speed of 10000 g and a temperature of 4° C. for 1 hour.
5) Every 40 g of the acquired bacteria were added with 1000 mL of extracting solution A (50 mM Tris, pH 8.0, containing 1.5 mm EDTA) to prepare a suspension, and then lysed with 250 mg of lysozyme.
6) A lysate was processed with an extracting solution B (1.5 M NaCl, 100 mM CaCl₂), 100 mM MgCl₂, 0.002% DNase I). Every 1000 mL of the above lysate was added with 100 mL of extracting solution B.
7) After the mixture was centrifuged at 4° C. and 10000 g for 10 minutes, precipitated inclusion bodies were collected and evenly suspended in 50 mM phosphate buffer (containing 0.15 M sodium chloride and 4 M urea, pH 7.0). 1 g of precipitates were added with 10 mL of buffer and centrifuged at 10000 g and 4° C. for 10 minutes, and precipitates were insoluble inclusion bodies, which could be collected and stored at −80° C. for half a year.

Folding and purification of protein:
1) Every 10 g of crude purified inclusion bodies were dissolved in 2000 mL of buffer (0.1 M Tris-HCl, pH 7.5 containing 6 M guanidine hydrochloride, 20 mM DTT, and 20 mM EDTA), and stirred at 20° C. for 1 hour.
2) The mixture was centrifuged at 10000 g and 4° C. for 30 minutes, and then a supernatant was taken for ultrafiltration and concentration. Every 2000 mL of the supernatant was concentrated into about 200 mL.
3) A concentrated sample passed through a Ni-NTA affinity column, and was purified on an AKTA protein purification system. Specific operation steps were the same as those in the above description.
4) The collected sample was dialyzed in a chromatographic cabinet at 4° C. according to the above method.
5) The dialyzed sample was centrifuged at 10000 g and 4° C. for 30 minutes, and then a supernatant was taken.
6) The supernatant was concentrated by an ultrafiltration concentrator, and every 2000 mL of the supernatant was concentrated into 300 mL.
7) The sample was purified by Sephadex G-75 chromatography on the AKTA protein purification system, with a column length of 1.2 m, a diameter of 4 cm, and a flow rate of 1 mL/min.
8) A sample was collected according to a protein peak of the AKTA protein purification system to obtain a purified fusion protein.

Sterilization and endotoxin removal of protein
1) An endotoxin of the purified fusion protein was removed by a Polymyxin (Bio-rad) chromatographic column.
2) An endotoxin-removed sample was subjected to filtration sterilization by a 0.22 μm sterile filter, and then subpackaged and stored at 4° C.

The present invention provides an idea for a betacoronavirus fusion protein and a preparation method thereof, with many methods and ways to realize the technical solution of the present invention specifically. Those described above are merely the preferred embodiments of the present invention, and it should be pointed out that those of ordinary skills in the art may further make improvements and decorations without departing from the principle of the present invention, and these improvements and decorations should also be regarded as the scope of protection of the present invention. All the unspecified components in the embodiments can be realized by the prior art.

1. A beta-coronavirus fusion recombinant protein having the amino acid sequence shown by SEQ ID NO. 1.

2. The beta-coronavirus fusion recombinant protein according to claim 1, wherein the beta-coronavirus fusion recombinant protein is encoded by a nuclei acid having the nucleotide sequences of SEQ ID NO. 2.

3. The beta-coronavirus fusion recombinant protein according to claim 2, wherein the nuclei acid is incorporated into an expression vector to become as a recombinant expression vector that expresses the beta-coronavirus fusion recombinant protein.

4. The beta-coronavirus fusion recombinant protein according to claim 3, wherein the recombinant expression vector is transformed into a bacterium to generate a recombinant bacterium.

5. The beta-coronavirus fusion recombinant protein according to claim 3, wherein the beta-coronavirus fusion recombinant protein is prepared by the following steps:
(1) using a full-length DNA of SARS-COV-2 as a template, designing different PCR primers for a COVID19-SF2 protein fragment and a COVID19-SF5 protein fragment, introducing a BamH I enzyme cutting site at a 5′ terminal of the COVID19-SF2 protein fragment and introducing a reverse complementary sequence of a flexible linker peptide at a 3′ terminal of the COVID19-SF2 protein fragment, and introducing a sequence of the flexible liner peptide at a 5′ terminal of the COVID19-SF5 protein fragment, introducing a Hind III enzyme cutting site at a 3′ terminal of the COVID19-SF5 protein fragment and introducing a 6×His encoding gene at a C terminal of the COVID19-SF5 protein to obtain PCR products of the COVID19-SF2 protein fragment and the COVID19-SF5 protein fragment, wherein an amino acid sequence of the COVID19-SF2 fragment is a 305^{th} amino acid to a 525^{th} amino acid of an S protein of a novel coronavirus COVID-19, and an amino acid sequence of the COVID19-SF5 fragment is an 880^{th} amino acid to a 1084^{th} amino acid of the S protein of the novel coronavirus COVID-19;
(2) carrying out overlap extension PCR on the PCR products of the COVID19-SF2 protein fragment and the COVID19-SF5 protein fragment respectively amplified in the first step, and obtaining a fusion protein expression gene by ligation;
(3) ligating the fusion protein expression gene with an expression vector pQE-3 through the BamH I and Hind III enzyme cutting sites at the 5′ terminal and the 3′ terminal of the sequences to obtain an expression vector containing the fusion protein gene;
(4) transforming the expression vector containing the fusion protein gene into an Escherichia coli M15 strain by a competent cell preparation method;
(5) selecting strains growing on a selective plate for colony PCR, and inducing protein expression of strains positive for PCR;
(6) taking clones with positive colony PCR for expanded culture, and inducing expression with IPTG; and
(7) acquiring inclusion bodies for purification and folding to obtain the recombinant fusion protein.

6. An industrial preparation method of the fusion recombinant protein according to claim 4, comprising the following steps of:
(1) using the recombinant bacterium as a seed strain, and amplifying the seed strain by overnight shaking as a seed solution;
(2) subjecting the seed solution to fermentation culture in a 2×YT culture medium, collecting fermented bacteria by an industrial automatic continuous centrifuge, preparing the collected bacteria into a suspension with an extracting solution A, lysing with a hydrolyase, then processing with an extracting solution B, collecting precipitated inclusion bodies after centrifugation, diluting with a buffer, and then centrifuging, wherein precipitates are insoluble inclusion bodies;
(3) further dissolving the inclusion bodies in a buffer, centrifuging and collect ing supernatant for ultrafiltration and concentration to obtain a concentrated sample, then allowing the concentrated sample to pass through a Ni-NTA affinity column, purifying on an AKTA protein purification system, and collecting the sample; and
(4) dialyzing the collected sample in a chromatographic cabinet at 4° C., centrifuging the dialyzed sample to take a supernatant, concentrating the supernatant by an ultrafiltration concentrator, and then purifying by Sephadex G-75 chromatography on the AKTA protein purification system; and collecting a sample according to a protein peak of the AKTA protein purification system to obtain a purified fusion protein.

7. The method according to claim 6, wherein fermentation conditions comprise: a dissolved oxygen value or dissolved oxygen concentration (DO value) of 60%, a temperature of 37° C., a pH value of 7.0, the addition of an inducer IPTG when a bacterial concentration reaches a peak value, and total culture time of 7 hours.

8. A serum comprehensive antibody IgG of the beta-coronavirus fusion recombinant of claim 1, wherein an antiserum is obtained from a mouse immunized with the beta-coronavirus recombinant fusion protein, and the serum comprehensive antibody IgG is obtained by purification.

9. A method for preparing a vaccine comprising a step of using the beta-coronavirus fusion recombinant protein of claim 1 as an antigen.
